(19) United States
(12) Patent Application Publication
(10) Pub. No.: US 2008/0167364 A1
(43) Pub. Date: Jul. 10, 2008

(54) RAMIPRIL-AMINE SALTS

(75) Inventors: Paul Jonathan Harrison, Dublin (IE); Anna Marie Elizabeth Power, Dublin (IE); Deirdre O'Keeffe, London (GB)

Correspondence Address: WILSON SONSINI GOODRICH & ROSATI, 650 PAGE MILL ROAD, PALO ALTO, CA, 94304-1050, US

(73) Assignee: Selamine Limited, Dublin (IE)

(21) Appl. No.: 11/948,049

(22) Filed: Nov. 30, 2007

(30) Foreign Application Priority Data
Dec. 1, 2006 (GB) 0624090.7

(51) Int. Cl. A61K 31/403; C07D 209/52; A61P 9/00; A61P 9/10; A61P 3/10

(52) U.S. Cl. 514/412; 548/452

(57) ABSTRACT

The present invention relates to ramipril-amine salts, such as, primary, secondary, tertiary and quaternary salts of ramipril.

### FIELD OF THE INVENTION

The present invention relates to ramipril-amine salts, such as, primary, secondary, tertiary and quaternary salts of ramipril.

### INCORPORATION BY REFERENCE

Each of the references cited is hereby expressly incorporated herein by reference.

### BACKGROUND OF THE INVENTION

Ramipril, the United States Adopted Name (USAN) for (2S,3aS,6aS)-1[(S)-N-[(S)-1-carboxy-3-phenylpropyl]alanyl]octahydrocyclopenta[b]pyrrole-2-carboxylic acid, 1-ethyl ester (CAS Number 087333-19-5) is an angiotensin converting enzyme (ACE) inhibitor having the chemical structure shown below (I).

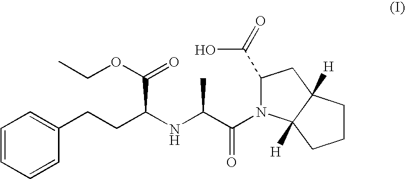

Ramipril has been used for the treatment of hypertension, heart failure, stroke, myocardial infarction, diabetes and cardiovascular disease. It is commercially available at 1.25 mg, 2.5 mg, 5 mg, 10 mg and 15 mg strengths.

Degradation of pharmaceutically active compounds is of concern to both medical practitioners and to the community at large. If significant degradation takes place between manufacture and administration of an active then suboptimal dosing is highly likely. For actives used in the treatment of hypertension and cardiovascular disease dosing accuracy is of tantamount importance as ineffective treatment is likely to result in life-threatening complications.

It would be useful to provide a form of ramipril, that provides benefits over current formulations of ramipril, for example, a form of ramipril that avoids significant degradation to inactive impurities.

An object of the present invention is to provide a form of ramipril, that avoids significant degradation to inactive impurities.

### SUMMARY OF THE INVENTION

The present invention relates to a ramipril-amine salt. A ramipril-amine salt is useful for the treatment or prevention of a cardiovascular disorder, renal failure, an ischemic condition, diabetes mellitus or a diabetic complication, or stroke (each being a “Condition”)

In another embodiment, the present invention relates to compositions comprising a therapeutically or prophylactically effective amount of a ramipril-amine salt and a pharmaceutically acceptable carrier. The compositions are useful for treating or preventing a Condition.

In yet another embodiment, the invention relates to methods for treating or preventing a Condition, comprising administering to a subject in need thereof a therapeutically or prophylactically effective amount of a ramipril-amine salt.

In still yet another embodiment, the invention relates to methods for reducing the incidence of recurrence or severity of a symptom of a Condition, comprising administering to a subject in need thereof a therapeutically or prophylactically effective amount of a ramipril-amine salt.

### BRIEF DESCRIPTION OF THE DRAWINGS

FIG. 1 is a Form 1 X-Ray Powder diffractogram of a ramipril-erbumine salt.

FIG. 2 is an FTIR spectrum of a ramipril-erbumine salt.

FIG. 3 is an X-Ray Powder diffractogram of a ramipril-ammonia salt.

FIG. 4 is an FTIR spectrum of a ramipril-ammonia salt.

FIG. 5 is a 1H-NMR spectrum of a ramipril-erbumine salt in d4-methanol.

FIG. 6 is a thermal gravimetric analysis (TGA) and differentially scanning calorimetry (DSC) scan of a ramipril-erbumine salt.

FIG. 7 is a 1H-NMR spectrum of a ramipril-ammonia salt in d4-methanol.

FIG. 8 is a thermal gravimetric analysis (TGA) and differentially scanning calorimetry (DSC) scan of a ramipril-ammonia salt.

FIG. 9 is a Form 2 X-Ray Powder diffractogram of a ramipril-erbumine salt.

FIG. 10 is a 1H-NMR spectrum of a Form 2 ramipril-erbumine salt in D₂O.

### DETAILED DESCRIPTION

According to the invention there is provided salts of ramipril, being primary, secondary, tertiary and quaternary amine ramipril salts; in certain embodiments salts of ramipril and an organic amine.

In certain embodiments, the organic amine is triethylamine, ethanolamine, triethanolamine, meglumine, ethylamine diamine, choline, procaine or benzathine. In certain embodiments the ramipril-amine salt is ramipril-choline, ramipril-meglumine or ramipril-procaine. In certain embodiments, the amine is not dicyclohexylamine or N-meglumine.

In other embodiments, the invention relates to a ramipril-amine salt of formula X(NR¹R²R³), wherein X is ramipril and each of R¹, R² and R³ is independently selected from H, unsubstituted or substituted alkyl, unsubstituted or substituted cycloalkyl, unsubstituted or substituted aryl, unsubstituted or substituted heteroaryl, or unsubstituted or substituted heterocycloalkyl. In certain embodiments, each of R¹, R² and R³ is independently selected from H, unsubstituted or substituted alkyl, unsubstituted or substituted cycloalkyl, or unsubstituted or substituted aryl. In certain embodiments, R¹, R² and R³ are H or alkyl, provided that at least one of R¹, R² and R³ is not H. In still other embodiments, R¹, R² and R³ are H or unsubstituted alkyl, provided that at least one of R¹, R² and R³ is not H.

In still other embodiments, the ramipril-amine salt of the invention has the formula X(NR¹R²R³), wherein each of R¹, R² and R³ is independently selected from H and alkyl. In some embodiments, two of R¹, R² and R³ are H (primary amine salts); one of R¹, R² and R³ are H (secondary amine salts); or none of R¹, R² and R³ are H (tertiary amine salts). In other embodiments, each of R¹, R² and R³ are the same. In specific embodiments, the ramipril-amine salt of the invention has the formula X(NR¹R²R³), wherein one of R¹, R² and R³ is t-butyl and the other two are H.

In specific embodiments, the ramipril-amine salt of the invention has the formula X(NR¹R²R³), wherein all of R¹, R² and R³ are ethyl.

In certain embodiments, the ramipril-amine salt is a ramipril-erbumine salt, a ramipril-triethylamine salt or a ramipril-meglumine salt.

In the formula X(NR¹R²R³), it is to be understood that ramipril is in its conjugate base form and the nitrogen atom of NR¹R²R³ has a (+) charge.

In still other embodiments, the invention relates to a ramipril-amine salt of formula X(NR¹R²R³R⁴) wherein X is ramipril and each of R¹, R², R³ and R⁴ is independently selected from H, unsubstituted or substituted alkyl, unsubstituted or substituted cycloalkyl, unsubstituted or substituted aryl, unsubstituted or substituted heteroaryl, or unsubstituted or substituted heterocycloalkyl. In certain embodiments, each of R¹, R², R³, and R⁴ is independently selected from H, unsubstituted or substituted alkyl, unsubstituted or substituted cycloalkyl, or unsubstituted or substituted aryl. In certain embodiments, R¹, R², R³, and R⁴ are H or alkyl, provided that at least one of R¹, R², R¹, and R⁴ is not H. In still other embodiments, R¹, R², R³, and R⁴ are H or unsubstituted alkyl, provided that at least one of R¹, R², R³, and R⁴ is not H. In certain embodiments, (NR¹R²R³R⁴)⁺ is not dicyclohexylammonium or N-methyl-D-glucammonium.

In certain embodiments of a quaternary amine salt, each of R¹, R², R³, and R⁴ is independently selected from H and alkyl. In further embodiments, none of R¹, R², R³, and R⁴ is H, and in further embodiments still, all of R¹, R², R³, and R⁴ are the same.

In specific embodiments, the ramipril-amine salt is ramipril-ammonia salt.

In the formula X(NR¹R²R³R⁴), it is to be understood that ramipril is in its conjugate base form and the nitrogen atom of (NR¹R²R³R⁴) has a (+) charge.

Another embodiment of the invention relates to a salt of formula X (NR¹R²R³)₂, wherein X is ramipril in diacid form, also referred to as ramiprilat, and R¹, R² and R³ are as defined elsewhere herein.

In the formula X (NR¹R²R³)₂, it is to be understood that ramipril is in its diacid conjugate base form and the nitrogen atom of each (NR¹R²R³) has a (+) charge.

In one embodiment, the ramipril-amine salt is substantially free of ramipril (free acid) or amine (free base). In this context, the term “substantially free” means that the ramipril-amine salt comprises no more than 5% by weight of ramipril (free acid) or amine (free base); in another embodiment, no more than 2% by weight of ramipril (free acid) or amine (free base); in still another embodiment, no more than 1% by weight of ramipril (free acid) or amine (free base); in yet another embodiment no more than 0.5% by weight of ramipril (free acid) or amine (free base); in still yet another embodiment no more than 0.1% by weight of ramipril (free acid) or amine (free base). In another embodiment, the ramipril-amine salt is substantially free of ramipril (free acid) and amine (free base). In this context, the term “substantially free” means that the ramipril-amine salt comprises no more than 5% by weight of ramipril (free acid) and amine (free base); in another embodiment, no more than 2% by weight of ramipril (free acid) and amine (free base); in still another embodiment, no more than 1% by weight of ramipril (free acid) and amine (free base); in yet another embodiment no more than 0.5% by weight of ramipril (free acid) and amine (free base); in still yet another embodiment no more than 0.1% by weight of ramipril (free acid) and amine (free base).

As the amine selected may possess one or more chiral centers, in one embodiment, a ramipril-amine salt of the invention utilize one or more enantiomers of the amine, diasteriomers of the amine or mixtures (racemic or otherwise) of the amine.

A ramipril-amine salt of is believed to offer the potential for alternatives to existing ramipril formulations and potential benefits include, but are not limited to improved solubility, dissolution and/or hygroscopicity. Physical and/or chemical stability may be improved, and a ramipril-amine salt may have improved flowability and/or improved compressibility—relevant in tablet manufacture.

### DEFINITIONS

In order that the invention may be more readily understood, certain terms are first defined and collected here for convenience. Other definitions appear in context throughout the application.

The term “alkyl” refers to a straight or branched hydrocarbon chain radical, having solely carbon and hydrogen atoms, having in the range from one up to eight carbon atoms, and which is attached to the rest of the molecule by a single bond, such as illustratively, methyl, ethyl, n-propyl 1-methylethyl (iso-propyl), n-butyl, n-pentyl, and 1,1-dimethylethyl (tert-butyl).

The term “cycloalkyl” denotes a non-aromatic mono or multicyclic ring system having in the range of 3 up to 14 carbon atoms such as cyclopropyl, cyclobutyl, cyclopentyl, cyclohexyl and cycloheptyl. Examples of multicyclic cycloalkyl groups include decahydronapththyl. Examples of bridged cycloalkyl groups or sprirobicycloalkyl groups include adamantyl norbornyl, and spiro[4.4]nonyl groups.

The term “aryl” refers to a monocyclic or bicyclic aromatic radical having in the range of 6 up to 14 carbon atoms such as phenyl, naphthyl, indanyl, and biphenyl.

The term “heteroaryl” refers to a 5- to 13-membered monocyclic, bicyclic or tricyclic aromatic heterocycle having in the range of from 1 up to 4 heteroatoms of nitrogen, phosphorus, oxygen and sulfur, which ring or ring system can be linked via a carbon atom or a nitrogen atom, if such an atom is present. For purposes of this invention, the heteroaryl ring radical may be a monocyclic, bicyclic or tricyclic ring system. Examples of such heteroaryl radicals are: pyridyl, pyridyl N-oxide, pyrimidyl, pyridazinyl, pyrazinyl, thienyl, furyl, pyrrolyl, pyrazolyl, imidazolyl, thiazolyl, oxazolyl or isoxazolyl, indolicenyl, indolyl, benzo[b]thienyl, benzo[b]furyl, benzothiazolyl, benzothiadiazolyl, indazolyl, quinolyl, isoquinolyl, isoquinolyl, naphthyridinyl, quinazolinyl, oxadiazolyl, benzoxazolyl, tetrazoyl, triazolyl, thiadiazolyl, and benzimidazolyl.

The term “heterocycloalkyl” refers to a 3- to 13-membered saturated or unsaturated heterocycle having in the range from 1 up to 4 heteroatoms of nitrogen, phosphorus, oxygen and sulfur, which ring or ring system can be linked via a carbon atom or a nitrogen atom, if such an atom is present. For purposes of this invention, the heterocycle radical may be a monocyclic, bicyclic or tricyclic ring system, which may include fused, bridged or spiro ring systems. Examples of such heterocycles are: tetrahydropyranyl, aziridyl, azepanyl, tetrahydrofuryl, pyrrolidinyl, pyrrolinyl, piperidinyl, 1,2 dihydropyridinyl, 1,4 dihydropyridinyl, piperazinyl, morpholinyl, thiomorpholinyl, azepinyl, oxazolinyl, thiazolinyl and 1,4 diazepinyl.

In certain embodiments, the alkyl, cycloalkyl, aryl, heteroaryl or heterocycloalkyl group is further substituted. Illustrative substitutents include, but are not limited to straight or branched alkyl having 1-4 carbon atoms, nitro, halogen and hydroxy. Unless indicated otherwise, the alkyl, cycloalkyl, aryl, heteroaryl or heterocycloalkyl group is unsubstituted.

The term “erbumine” means tert-butylamine.

The term “meglumine” means N-methylglucamine.

A “therapeutically effective amount” of a ramipril-amine salt is an amount that is effective to treat a Condition.

A “prophylactically effective amount” of a ramipril-amine salt is an amount that is effective to prevent a Condition.

The term “subject” refers to an animal such as a mammal, including, but not limited to, a primate (e.g., a human), a cow, a sheep, a goat, a horse, a dog, a cat, a rabbit, a rat, a mouse and the like. In certain embodiments, the subject is a human.

It is also to be understood that the terminology used herein is for purposes of describing particular embodiments only, and is not intended to be limiting. As used in the specification and the appended claims, the singular forms “a”, “an”, and “the” include plural referents unless the context clearly indicates otherwise.

Synthesis of Ramipril-Amine Salt

To provide ramipril-amine salts of the invention, ramipril (free acid) and a solvent are mixed, optionally with heating, until all solids have been suspended or dissolved. Ramipril (free acid) is commercially available or can be prepared by the methods described in U.S. Pat. No. 4,587,258; 5,061,772 or 6,407,262. Solvents which may be utilized include, but are not limited to, toluene; alcohols such as methanol, ethanol, propan-2-ol and propanol; esters such as ethyl acetate; ketones such as acetone and butanone; halogenated hydrocarbons such as dichloromethane; and ethers such as tetrahydrofuran (THF) and diethyl ether. The suspension or solution is generally heated with stirring from about 30° C. to about 75° C. (depending on the solvent employed) for approximately 1-60 minutes; in some embodiments from 15-45 minutes; in other embodiments from 25-35 minutes.

After this time the suspension, if any, is heated again to about 30° C. to about 90° C. (depending on the solvent employed) until all solids dissolve. After about 5 minutes, an amine is added (from about 1.0 equivalents to about 5.0 equivalents; in some embodiments from about 1.0 equivalents to about 2.5 equivalents; in still other embodiments from about 1.0 equivalents to about 1.5 equivalents; in some other embodiments from about 0.5 equivalents to about 1.0 equivalents; and in still other embodiments from about 0.75 equivalents to about 1.0 equivalents) was added, optionally in a solvent which may be the same as or different than the initial solvent employed in the suspension or solution.

After formation of a precipitate, the mixture is generally cooled to about 25° C.; in some embodiments to about 15° C.; in still other embodiments to about 10° C.; and in other embodiments to about 5° C. to. The precipitate is filtered (optionally under vacuum), washed, and dried (either air dried, oven dried at ambient pressure or oven dried under reduced pressure).

In certain aspects of the invention the ramipril-amine salt is provided in non-crystalline form, taking the form e.g. of a solid or oil. As an oil, a ramipril-amine salt can be adsorbed onto or admixed with a pharmaceutically acceptable carrier for use in a pharmaceutical composition. In further aspects of the invention, a ramipril-amine salt is provided in crystalline form.

Treatment or Prevention of a Condition

In accordance with the invention, a ramipril-amine salt is useful for the treatment or prevention of a Condition as set forth below.

Treatment or Prevention of a Cardiovascular Disorder

A ramipril-amine salt is useful for treating or preventing a cardiovascular disorder. Examples of cardiovascular disorder include, but are not limited to, hypertension, congestive heart failure (such as chronic or acute heart failure), atherosclerosis, hypercholesterolemia, circulatory shock, cardiomyopathy, cardiac transplant, myocardial infarction, and a cardiac arrhythmia, such as atrial fibrillation, supraventricular tachycardia, atrial flutter, and paroxysmal atrial tachycardia.

In one embodiment, the cardiovascular disorder is chronic heart failure.

In another embodiment, the cardiovascular disorder is acute heart failure.

In yet another embodiment, the cardiovascular disorder is cardiac arrhythmia. In still another embodiment, the cardiac arrhythmia is atrial fibrillation, supraventricular tachycardia, atrial flutter or paroxysmal atrial tachycardia.

In one embodiment, the cardiovascular disorder is chronic heart failure, atrial fibrillation, supraventricular tachycardia, atrial flutter or paroxysmal atrial tachycardia.

Treatment or Prevention of an Ischemic Condition

A ramipril-amine salt is useful for treating or preventing an ischemic condition. Examples of ischemic conditions include, but are not limited to, stable angina, unstable angina, myocardial ischemia, hepatic ischemia, mesenteric artery ischemia, ischemic heart disease, intestinal ischemia, critical limb ischemia, chronic critical limb ischemia, cerebral ischemia, acute cardiac ischemia, and an ischemic disease of the central nervous system, such as stroke or cerebral ischemia.

In one embodiment, the ischemic condition is myocardial ischemia, stable angina, unstable angina, stroke, ischemic heart disease or cerebral ischemia.

Treatment or Prevention of Renal Failure

A ramipril-amine salt is useful for treating or preventing renal failure.

In one embodiment, the renal failure is chronic renal failure. In another embodiment, the renal failure is acute renal failure.

Treatment or Prevention of Diabetes Mellitus or a Diabetic Complication

A ramipril-amine salt is useful for treating or preventing diabetes mellitus or one or more of its complications. Examples of diabetes mellitus include, but are not limited to, Type I diabetes (Insulin Dependent Diabetes Mellitus), Type II diabetes (Non-Insulin Dependent Diabetes Mellitus), gestational diabetes, autoimmune diabetes, insulinopathies, diabetes due to pancreatic disease, diabetes associated with other endocrine diseases (such as Cushing's Syndrome, acromegaly, pheochromocytoma, glucagonoma, primary aldosteronism or somatostatinoma), Type A insulin resistance syndrome, Type B insulin resistance syndrome, lipatrophic diabetes, and diabetes induced by [beta]-cell toxins. A ramipril-amine salt is also useful for treating or preventing a complication of diabetes mellitus. Examples of complications of diabetes mellitus include, but are not limited to, diabetic cataract, glaucoma, retinopathy, nephropathy (such as microalbuminuria or progressive diabetic nephropathy), polyneuropathy, gangrene of the feet, immune-complex vasculitis, systemic lupus erythematosus (SLE), atherosclerotic coronary arterial disease, peripheral arterial disease, nonketotic hyperglycemic-hyperosmolar coma, mononeuropathies, autonomic neuropathy, foot ulcers, joint problems, and a skin or mucous membrane complication (such as an infection, a shin spot, a candidal infection or necrobiosis lipoidica diabeticorumobesity), hyperlipidemia, hypertension, syndrome of insulin resistance, coronary artery disease, retinopathy, neuropathy (such as diabetic neuropathy, polyneuropathy or mononeuropathy), autonomic neuropathy, a foot ulcer, a joint problem, a fungal infection, cardiomyopathy, and a bacterial infection. In one embodiment diabetes mellitus is Type I diabetes mellitus or Type II diabetes mellitus.

Reduction of Incidence of Recurrence or Severity of Symptoms Associated with a Condition

A ramipril-amine salt is useful for the reduction of the incidence of recurrence or the severity of a symptom associated with a Condition.

In certain embodiments, a ramipril-amine salt is useful for the reduction of the incidence of recurrence of heart attack.

In certain embodiments, a ramipril-amine salt is useful for the reduction of the incidence of recurrence of hypertension. In other embodiments, a ramipril-amine salt is useful for the reduction of the severity of symptoms associated with hypertension.

In certain embodiments, a ramipril-amine salt is useful for the reduction of the incidence of recurrence of stroke. In still other embodiments, a ramipril-amine salt is useful for the reduction of cognitive impairment associated with stroke.

Formulation and Administration

Due to their activity, a ramipril-amine salt is advantageously useful in veterinary or human medicine. As described above, a ramipril-amine salt is useful for treating or preventing a Condition in a subject in need thereof.

A ramipril-amine salt can be administered in an amount that is effective to treat or prevent a Condition in a subject in need thereof.

When administered to a subject, a ramipril-amine salt can be administered as a component of a composition that comprises a pharmaceutically acceptable carrier. The present compositions, which comprise a ramipril-amine salt, can be administered orally. A ramipril-amine salt can also be administered by any other convenient route, for example, by infusion or bolus injection, by absorption through epithelial or mucocutaneous linings (e.g., oral, rectal, or intestinal mucosa) and can be administered together with another biologically active agent. Administration can be systemic or local. Various delivery systems are known, e.g., encapsulation in liposomes, microparticles, microcapsules and capsules.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, specifically to the ears, nose, eyes, or skin. In some instances, administration will result in the release of a ramipril-amine salt into the bloodstream.

In one embodiment, a ramipril-amine salt is administered orally. In other embodiments, it can be desirable to administer a ramipril-amine salt locally. This can be achieved, for example, and not by way of limitation, by local infusion during surgery, topical application, e.g., in conjunction with a wound dressing after surgery, by injection, by means of a catheter, by means of a suppository or enema, or by means of an implant, said implant being of a porous, non-porous, or gelatinous material, including membranes, such as sialastic membranes, or fibers.

In certain embodiments, it can be desirable to introduce a ramipril-amine salt into the central nervous system or gastrointestinal tract by any suitable route, including intraventricular, intrathecal, and epidural injection, and enema. Intraventricular injection can be facilitated by an intraventricular catheter, for example, attached to a reservoir, such as an Ommaya reservoir.

Pulmonary administration can also be employed, e.g., by use of an inhaler of nebulizer, and formulation with an aerosolizing agent, or via perfusion in a fluorocarbon oar, synthetic pulmonary surfactant. In certain embodiments, a ramipril-amine salt can be formulated as a suppository, with traditional binders and excipients such as triglycerides.

In another embodiment a ramipril-amine salt can be delivered in a vesicle, specifically a liposome (see Langer, Science 249:1527-1533 (1990) and Treat or prevent et al, Liposomes in Therapy of Infectious Disease and Cancer 317-327 and 353-365 (1989)).

In yet another embodiment, a ramipril-amine salt can be delivered in a controlled-release system or sustained-release system {see, e.g., Goodson, in Medical Applications of Controlled Release, supra, vol. 2, pp. 115-138 (1984)). Other controlled or sustained-release systems discussed in the review by Langer, Science 249: 1527-1533 (1990) can be used. In one embodiment a pump can be used (Langer, Science 249; 1527-1533 (1990); Sefton, CRC Crit. Ref. Biomed. Eng. 14:201 (1987); Buchwald et al, Surgery 88:507 (1980); and Saudek et al., N. Engl. J Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sd. Rev. Macromol. Chem. 2:61 (1983); Levy et al, Science 228:190 (1935); During et al, Ann. Neural. 25:351 (1989); and Howard et al, J. Neurosurg. 71:105 (1989)).

Dosage Regimen

Suitable dosages and formulations of a ramipril-amine salt can be empirically determined those of skill in the art. Standard texts, such as Remington: The Science and Practice of Pharmacy, 17th edition, Mack Publishing Company, and the Physician's Desk Reference, each of which are incorporated herein by reference, can be consulted to prepare suitable compositions and doses for administration. A determination of the appropriate dosage is within the skill of one in the art given the parameters for use described herein.

Standard texts, such as Remington: The Science and Practice of Pharmacy, 17th edition, Mack Publishing Company, incorporated herein by reference, can be consulted to prepare suitable compositions and formulations for administration, without undue experimentation. Suitable dosages can also be based upon the text and documents cited herein. A determination of the appropriate dosages is within the skill of one in the art given the parameters herein.

The dosage regimen utilizing a ramipril-amine salt can be selected in accordance with a variety of factors including type, species, age, weight, sex and medical condition of the subject; the severity of the Condition to be treated; the route of administration; the renal or hepatic function of the subject; and the specific ramipril-amine salt employed. A ramipril-amine salt can be administered in a single daily dose, or the total daily dosage can be administered in divided doses of two, three or four times daily. Furthermore, a ramipril-amine salt can be administered in intranasal form via topical use of suitable intranasal carriers, or via transdermal routes, using those forms of transdermal skin patches known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of a ramipril-amine salt ranges from about 0.1% to about 15%, w/w or w/v. A ramipril-amine salt can be assayed in vitro or in vivo for the desired therapeutic or prophylactic activity prior to use in humans. Animal model systems can be used to demonstrate safety and efficacy in humans.

The amount of a ramipril-amine salt that is effective in the treatment or prevention of a Condition can be determined using standard clinical techniques. In addition, in vitro or in vivo assays can optionally be employed to help identify optimal dosage ranges. The precise dose to be employed can also depend on the route of administration, and the seriousness of the Condition being treated and can be decided according to the judgment of the practitioner and each subject's circumstances in view of, e.g., published clinical studies. Suitable effective dosage amounts, however, range from about 1 mg to about 15,000 mg per day; about 1000 mg to about 10,000 mg per day; or about 2,500 mg to about 5,000 mg per day. The dosage of ramipril salts can range from about 150 to 1500 mg/kg of body weight. Such dosages may vary, for example, depending on whether multiple administrations are given, tissue type and route of administration, the Condition of the individual, the desired objective and any other factors known to those of skill in the art. Administrations can be conducted infrequently, or on a regular weekly basis until a desired, measurable parameter is detected, such as diminution of disease symptoms. Administration can then be diminished, such as to a biweekly or monthly basis, as appropriate. The effective dosage amounts described herein refer to total amounts administered; that is, if more than one dose of a ramipril-amine salt is administered, the effective dosage amounts correspond to the total amount administered.

Compositions and Dosage Forms

The invention also provides a composition, comprising an effective amount a ramipril-amine salt described herein and a pharmaceutically acceptable diluent or carrier. In certain embodiments, the ramipril-amine salt is administered to the subject in a pharmaceutically acceptable formulation. In certain embodiments, the pharmaceutical compositions are suitable for topical, intravenous, parental, or oral administration. The methods of the invention further include administering to a subject an effective amount of a ramipril-amine salt in combination with another pharmaceutically active compound. Pharmaceutically active compounds that may be used can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference.

Methods of preparing these compositions can include the step of bringing into association a ramipril-amine salt with the carrier and, optionally, one or more accessory ingredients. These compositions may also contain adjuvants such as preservatives, wetting agents, emulsifying agents and dispersing agents.

Oral Dosage Forms

Ramipril-amine salts and compositions comprising them that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of a ramipril-amine salt or another therapeutic or prophylactic agent, and may be prepared by methods of pharmacy well known to those skilled in the art. See generally, Remington's Pharmaceutical Sciences, 18th ed., Mack Publishing, Easton Pa. (1990).

Typical oral dosage forms of the invention can be prepared by combining a ramipril-amine salt or another therapeutic or prophylactic agent(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules can be advantageous oral dosage unit forms, in which case solid excipients can be employed. If desired, tablets can be coated by standard aqueous or nonaqueous techniques. Such dosage forms can be prepared by any of the methods of pharmacy. In general, pharmaceutical compositions and dosage forms can be prepared by uniformly and intimately admixing a ramipril-amine salt or another therapeutic or prophylactic agent with liquid carriers, finely divided solid carriers, or both, and then shaping the product into the desired presentation if necessary.

For example, a tablet can be prepared by compression or molding. Compressed tablets can be prepared by compressing in a suitable machine a ramipril-amine salt or another therapeutic or prophylactic agent in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegants can be used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of a ramipril-amine salt or another therapeutic or prophylactic agent can be used to form solid oral dosage forms of the invention. The amount of disintegrant used, if any, varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W. R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

Parenteral and Intravascular Dosage Forms

A parenteral or intravascular dosage form can be administered to a subject via various routes including, but not limited to, subcutaneous, intravenous (including bolus injection and constant infusion), intramuscular, and intraarterial. Because their administration can bypass a subject's natural defenses against contaminants, parenteral and intravascular dosage forms can be, in general, sterile or capable of being sterilized prior to administration to a subject. Examples of parenteral dosage forms include, but are not limited to, solutions ready for injection, dry products (including, but not limited to lyophilized powders, pellets, and tablets) ready to be dissolved or suspended in a pharmaceutically acceptable carrier for injection, suspensions ready for injection, and emulsions.

Suitable carriers that can be used to provide parenteral dosage forms of the invention are known to those skilled in the art. Examples include, but are not limited to: Water for Injection USP; aqueous carriers such as, but not limited to, Sodium Chloride Injection, Ringer's Injection, Dextrose Injection, Dextrose and Sodium Chloride Injection, and Lactated Ringer's Injection; water-miscible carriers such as, but not limited to, ethyl alcohol, polyethylene glycol, and polypropylene glycol; and non-aqueous carriers such as, but not limited to, corn oil, cottonseed oil, peanut oil, sesame oil, ethyl oleate, isopropyl myristate, and benzyl benzoate.

Compounds that increase the solubility of a ramipril-amine salt or of another therapeutic or prophylactic agent disclosed herein can also be incorporated into the parenteral dosage forms of the invention.

For intravascular administration, for instance by direct injection into the blood vessel, or surrounding area, it may be desirable to administer the compositions locally to the area in need of treatment. This can be achieved, for example, by local infusion during surgery, by injection, by means of a catheter, or by means of an implant, said implant being of a porous, non-porous, or gelatinous material, including membranes, such as silastic membranes, or fibers.

Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of a ramipril-amine salt or another therapeutic or prophylactic agent.

Suitable excipients (e.g., carriers and diluents) and other materials that can be used to provide transdermal, topical, and mucosal dosage forms encompassed by this invention are known to those skilled in the art, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are known in the art. See, e.g., Remington's Pharmaceutical Sciences, 16th and 18th eds., Mack Publishing, Easton Pa. (1980 & 1990).

Additional components may be used prior to, in conjunction with, or subsequent to treatment with a ramipril-amine salt or another therapeutic or prophylactic agent of the invention. For example, penetration enhancers can be used to assist in delivering a ramipril-amine salt or another therapeutic or prophylactic agent to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 polysorbate 80) and Span 60 (sorbitan monostearate).

The pH of a pharmaceutical composition or dosage form, or of the tissue to which the pharmaceutical composition or dosage form is administered, may also be adjusted to improve delivery of a ramipril-amine salt or one or more other therapeutic or prophylactic agents. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of a ramipril-amine salt or one or more other therapeutic or prophylactic agents so as to improve delivery. In this regard, stearates can serve as a lipid carrier for the formulation, as an emulsifying agent or surfactant, and as a delivery-enhancing or penetration-enhancing agent. Different salts, hydrates or solvates of a ramipril-amine salt or one or more other therapeutic or prophylactic agents to further adjust the properties of the resultant composition.

Controlled-Release Dosage Forms

A ramipril-amine salt can be administered by controlled-release or sustained-release means or by delivery devices that are well known to those of skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of a ramipril-amine salt or one or more other therapeutic or prophylactic agents using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with a ramipril-amine salt or one or more other therapeutic or prophylactic agents of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of a ramipril-amine salt to treat or prevent a Condition over a period of time. Advantages of controlled- or sustained-release compositions include extended activity of the ramipril-amine salt, reduced dosage frequency, and increased subject compliance. In addition, controlled- or sustained-release compositions can favorably affect the time of onset of action or other characteristics, such as blood levels of a ramipril-amine salt, and can thus reduce the occurrence of adverse side effects, if any. Controlled- or sustained-release compositions can initially release an amount of a ramipril-amine salt that promptly produces the desired therapeutic or prophylactic effect, and gradually and continually release other amounts of a ramipril-amine salt to maintain this level of therapeutic or prophylactic effect over an extended period of time. To maintain a relatively constant level of a ramipril-amine salt in the body, a ramipril-amine salt can be released from the dosage form at a rate that will replace the amount of a ramipril-amine salt being metabolized and excreted from the body.

Controlled- or sustained-release of an a ramipril-amine salt or one or more other therapeutic or prophylactic agents can be stimulated by various conditions, including but not limited to, changes in pH, changes in temperature, concentration or availability of enzymes, concentration or availability of water, or other physiological conditions or compounds.

Combination Therapy

In certain embodiments, a ramipril-amine salt is administered to a subject concurrently with one or more other therapeutic or phrophylactic agents. For example, each component may be administered at about the same time or sequentially in any order at different points in time; however, if not administered at about the same time, they should be administered sufficiently closely in time so as to provide the desired treatment or preventative effect. In one embodiment, all components are administered at about the same time, and if not administered at about the same time, they are all administered on the same day, or within 1 hour, 2 hours, 6 hours, 12 hours, 48 hours or 72 hours of one another.

When used in combination with one or more other therapeutic or prophylactic agents, a ramipril-amine salt and the therapeutic or prophylactic agent can act additively or, in certain embodiments, synergistically. In one embodiment, a ramipril-amine salt or a composition of the invention is administered concurrently with another therapeutic or prophylactic agent in the same pharmaceutical composition. In another embodiment, a ramipril-amine salt or a composition of the invention is administered concurrently with another therapeutic or prophylactic agent in separate pharmaceutical compositions. In still another embodiment, a ramipril-amine salt or a composition of the invention is administered prior or subsequent to administration of another therapeutic or prophylactic agent. As some of the Conditions for which the compounds and compositions of the invention are useful in treating are chronic disorders, in one embodiment combination therapy involves alternating between administering a ramipril-amine salt or a composition of the invention and a pharmaceutical composition comprising another therapeutic or prophylactic agent, e.g., to minimize the toxicity associated with a particular drug. In certain embodiments, when a composition of the invention is administered concurrently with another therapeutic or prophylactic agent that potentially produces adverse side effects including, but not limited to toxicity, the therapeutic or prophylactic agent can advantageously be administered at a dose that falls below the threshold that the adverse side effect is elicited.

In one embodiment, the other therapeutic or prophylactic agent is a diuretic agent. Diuretic agents useful in the compositions and methods of the present invention include, but are not limited to, piretanide, amiloride, amiloride/HCTZ clorothiazide (Diuril® Oral Susp), bumetanide, clonidine/chlorthalidone (Clorpres®), chlorothalidone, deserpidine/methyclothiazide (Enduronyl-Forte®), chlorothiazide, ethacrynic acid (Edecrin®), fluorosemide, hydroflumethiazide (Saluron®), hydrochlorothiazide, polythiazide (Renese®), indapamide, prazosin/polythiazide (Minizide®), methyclothiazide, reserpine/methyclothiazide (Diutensin-R®), metolazone, spironolactone/hctz (Aldactazide 50/50®), torsemide, trichlormethazide (Naqua®), triamterene or triamterene/HCTZ.

In one embodiment, the other therapeutic or prophylactic agent is a statin. Statins useful in the compositions and methods of the present invention include, but are not limited to, atorvastating, cerivastatin, fluvastatin, lovastatin, pravastatin, rosuvastatin, or simvastatin.

In one embodiment, the other therapeutic or prophylactic agent is a calcium channel blocker. Calcium channel blockers useful in the compositions and methods of the present invention include, but are not limited to, amlodipine, bepridil, diltiazem, felodipine, isradipine, nicardipine, nifedipine, nimodipine, nisoldipine, or verapamil.

In one embodiment, the other therapeutic or prophylactic agent is an antiinflammatory agent. Anti-inflammatory agents useful in the compositions and methods of the present invention include but are not limited to non-steroidal anti-inflammatory agents (NSAIDs), such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

In one embodiment, the other therapeutic or prophylactic agent is an anti-renal failure agent. Anti-renal failure agents useful in the compositions and methods of the present invention include, but are not limited to, ACE (angiotensin-converting enzyme) inhibitors, such as captopril, enalaprilat, lisinopril, benazepril, fosinopril, trandolapril, quinapril, and ramipril; diuretics, such as mannitol, glycerin, furosemide, toresemide, tripamide, chlorothiazide, methyclothiazide, indapamide, amiloride, and spironolactone; and fibric acid agents, such as clofibrate, gemfibrozil, fenofibrate, ciprofibrate, and bezafibrate.

In one embodiment, the other therapeutic or prophylactic agent is an anti-diabetic agent. Anti-diabetic agents useful in the methods and compositions of the present invention include but are not limited to glucagons; somatostatin; diazoxide; sulfonylureas, such as tolbutamide, acetohexamide, tolazamide, chloropropamide, glybenclamide, glipizide, gliclazide, and glimepiride; insulin secretagogues, such as repaglinide, and nateglinide; biguanides, such as metformin and phenformin; thiazolidinediones, such as pioglitazone, rosiglitazone, and troglitazone; and [alpha]-glucosidase inhibitors, such as acarbose and miglitol.

In one embodiment, the other therapeutic or prophylactic agent is an anti-cardiovascular disease agent. Anti-cardiovascular disease agents useful in the methods and compositions of the present invention include but are not limited to carnitine; thiamine; and muscarinic receptor antagonists, such as atropine, scopolamine, homatropine, tropicamide, pirenzipine, ipratropium, tiotropium, and tolterodine.

In one embodiment, the other therapeutic or prophylactic agent is an antiemetic agent. Antiemetic agents useful in the methods and compositions of the present invention include, but are not limited to, metoclopromide, domperidone, prochlorperazine, promethazine, chlorpromazine, trimethobenzamide, ondansetron, granisetron, hydroxyzine, acetylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinol, thiethylperazine, thioproperazine, tropisetron, and mixtures thereof.

In one embodiment, the other therapeutic or prophylactic agent is an opioid analgesic agent. Opioid analgesic agents useful in the methods and compositions of the present invention include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorplianol, dextromethorphan, phenazocine, pentazocine, cyclazocine, methadone, isomethadone and propoxyphene.

In one embodiment, the other therapeutic or prophylactic agent is a non-opioid analgesic agent. Non-opioid analgesic agents useful in the methods and compositions of the present invention include, but are not limited to, aspirin, celecoxib, rofecoxib, diclofenac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In one embodiment, the other therapeutic or prophylactic agent is an antibiotic. Antibiotics useful in the methods and compositions of the present invention include, but are not limited to, a macrolide (e.g., tobramycin (Tobi®)), a cephalosporin (e.g., cephalexin (Keflex®), cephradine (Velosef®), cefiroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamecillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., firaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

In one embodiment, the other therapeutic or prophylactic agent is an antidepressant. Suitable antidepressants useful in the compositions and methods of the invention include, but are not limited to, binedaline, caroxazone, citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

In one embodiment, the other therapeutic or prophylactic agent is, an antifungal agent. Suitable antifungal agents useful in the compositions and methods of the invention in the compositions and methods of the invention include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofildin.

In one embodiment, the other therapeutic or prophylactic agent is an immunomodulatory agent. Immunomodulatory agents useful in the compositions and methods of the invention include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boemmger), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoramipril salt), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-114 (IDEC)) and CTLA4-immunoglobulin. Examples of cytokine receptor modulators include, but are not limited to, soluble cytokine receptors (e.g., the extracellular domain of a TNF-.alpha. receptor or a fragment thereof, the extracellular domain of an IL-1.beta. receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-.alpha., interferon (IFN)-.alpha., IFN-.beta., IFN-.gamma., and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-.alpha. antibodies, anti-IL-1.beta. antibodies, anti-IL-6 antibodies, anti-IL-8 antibodies (e.g., ABX-IL-8 (Abgenix)), and anti-IL-12 antibodies).

In one embodiment, the other therapeutic or prophylactic agent is a cytokine. Examples of cytokines useful in the compositions and methods of the invention include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PD GF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-alpha, and IFN-gamma).

In one embodiment, the other therapeutic or prophylactic agent is a hormone. Examples of hormones useful in the compositions and methods of the invention include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

In one embodiment, the other therapeutic or prophylactic agent is a β-interferon which include, but are not limited to, interferon β-1a and interferon β-1b.

Kits

This invention encompasses kits which, when used by, for example, a medical practitioner or subject, can simplify the administration of appropriate amounts of ramipril-amine salt to a subject.

A typical kit of the invention comprises one or more unit dosage forms of a ramipril-amine salt. In one embodiment, the kit comprises is a container, which can be sterile, containing an effective amount of a ramipril-amine salt and a physiologically acceptable carrier. The kit can further comprise a label or printed instructions instructing the use of a ramipril-amine salt to treat or prevent a Condition. The kit can also further comprise a unit dosage form of another prophylactic or therapeutic agent, for example, a container containing an effective amount of the other prophylactic or therapeutic agent. In one embodiment, the kit comprises a container containing an effective amount of a ramipril-amine salt and an effective amount of another prophylactic or therapeutic agent. Examples of other prophylactic or therapeutic agents include, but are not limited to, those listed above.

Kits of the invention can further comprise one or more devices that are useful to administer a ramipril-amine salt and/or another prophylactic or therapeutic agent. Examples of such devices include, but are not limited to, intravenous cannulation devices, syringes, drip bags, patches, topical gels, pumps, containers that provide protection from photodegredation, autoinjectors, and inhalers.

Kits of the invention can further comprise one or more pharmaceutically acceptable carriers that can be used to administer a ramipril-amine salt or another therapeutic or prophylactic agent. For example, if an a ramipril-amine salt or another therapeutic or prophylactic agent is provided in a solid form that must be reconstituted for parenteral administration, the kit can comprise a sealed container of a suitable carrier in which a ramipril-amine salt or another therapeutic or prophylactic agent can be dissolved or suspended to form a particulate-free sterile solution or suspension that is suitable for parenteral administration. Examples of pharmaceutically acceptable carriers include, but are not limited to: Water for Injection USP; aqueous carriers such as, but not limited to, Sodium Chloride Injection, Ringer's Injection, Dextrose Injection, Dextrose and Sodium Chloride Injection, and Lactated Ringer's Injection; water-miscible carriers such as, but not limited to, ethyl alcohol, polyethylene glycol, and polypropylene glycol; and non-aqueous carriers such as, but not limited to, corn oil, cottonseed oil, peanut oil, sesame oil, ethyl oleate, isopropyl myristate, and benzyl benzoate.

The invention is further described by way of the following non-limiting examples.

### EXAMPLES

In order that the invention may be more fully understood, the following examples are provided. It should be understood that these examples are for illustrative purposes only and are not to be construed as limiting the invention in any way.

Analytical Methods:

X-Ray Powder Diffraction

X-Ray Powder Diffraction patterns were collected using a Bruker AXS C2 GADDS diffractometer using Cu Kα radiation (40 kV, 40 mA), automated XYZ stage, laser video microscope for auto-sample positioning and a HiStar 2-dimensional area detector. X-ray optics include a single Göbel multilayer mirror coupled with a pinhole collimator of 0.3 mm.

The beam divergence, i.e., the effective size of the X-ray beam on the sample, was approximately 4 mm. A θ-θ continuous scan mode was employed with a sample-detector distance of 20 cm which gives an effective 2θ range of 3.2°-29.7°. Typically the sample was exposed to the X-ray beam for 120 seconds.

Samples run under ambient conditions were prepared as flat plate specimens using powder as received without grinding. Approximately 1-2 mg of the sample was lightly pressed on a glass slide to obtain a flat surface.

Nuclear Magnetic Resonance

NMR spectra were collected using a Bruker 400 MHz instrument equipped with an auto-sampler and controlled by a DRX400 console. Automated experiments were acquired using ICON-NMR v4.0.4 (build 1) running with Topspin v 1.3 (patch level 8) using the standard Bruker loaded experiments. For non-routine spectroscopy, data were acquired through the use of Topspin alone.

Samples were prepared in D₂O, d₆-DMSO or d₄-methanol, as indicated. Off-line analysis was carried out using ACD SpecManager v 9.09 (build 7703).

Differential Scanning Calorimetry

DSC data were collected on a Mettler DSC 823e equipped with a 50 position auto-sampler. The instrument was calibrated for energy and temperature using certified indium. Typically 0.5-3 mg of each sample, in a pin-holed aluminium pan, was heated at 10° C./minute from 25° C. to 300° C. A nitrogen purge at 50 mL/minute was maintained over the sample.

The instrument control and data analysis software was STARe v9.01.

Thermogravimetric Analysis

TGA data were collected using a Mettler TGA/SDTA 851e equipped with a 34 position auto-sampler. The instrument was temperature calibrated using certified indium. Typically 5-50 mg of each sample was loaded onto a pre-weighed aluminium crucible and was heated at 10° C.min−1 from ambient temperature to 350° C. A nitrogen purge at 50 mL/minute was maintained over the sample.

The instrument control and data analysis software was STARe v9.01.

Fourier Transform Infrared Spectroscopy

FT-IR data were collected using a Perkin-Elmer Spectrum One fitted with a Universal ATR sampling accessory.

The data were collected and analysed using Spectrum v5.0.1 software.

### SYNTHETIC EXAMPLES

Ramipril-Erbumine Salt

Method A

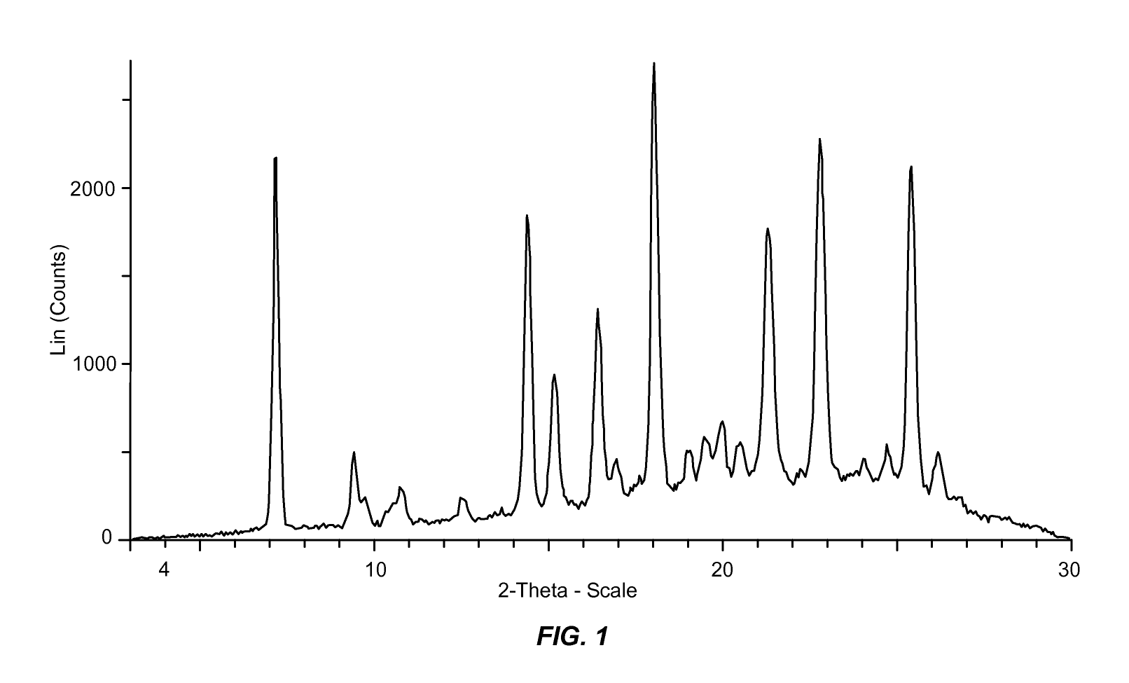

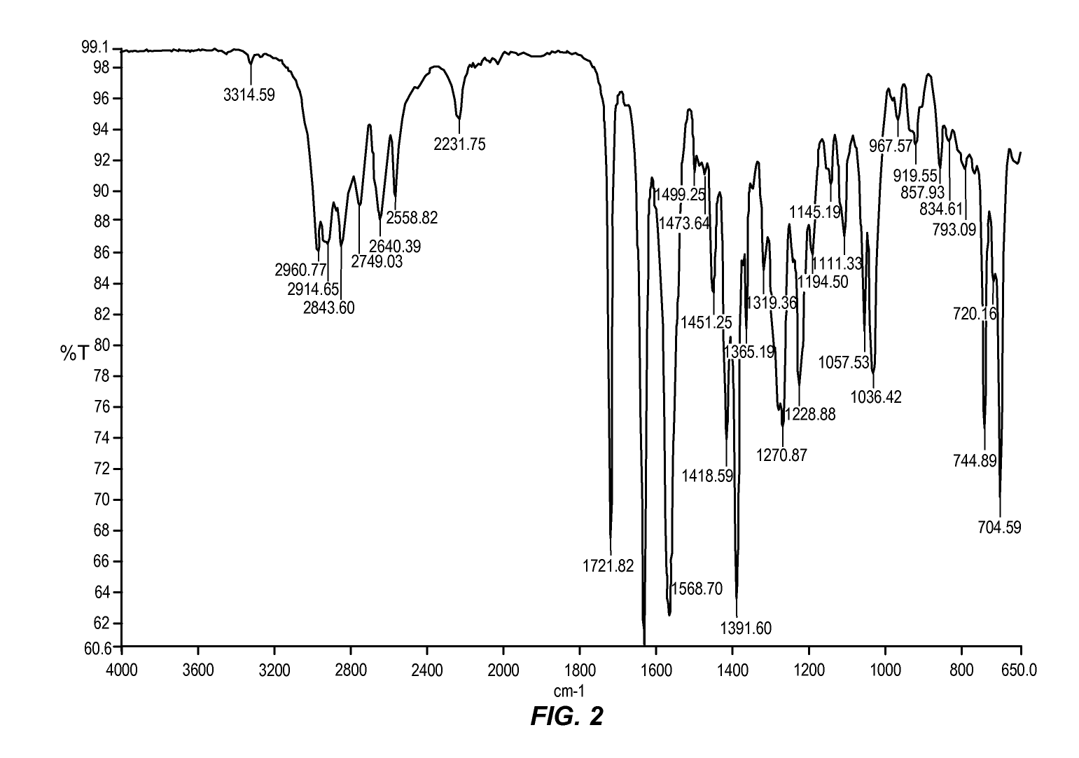

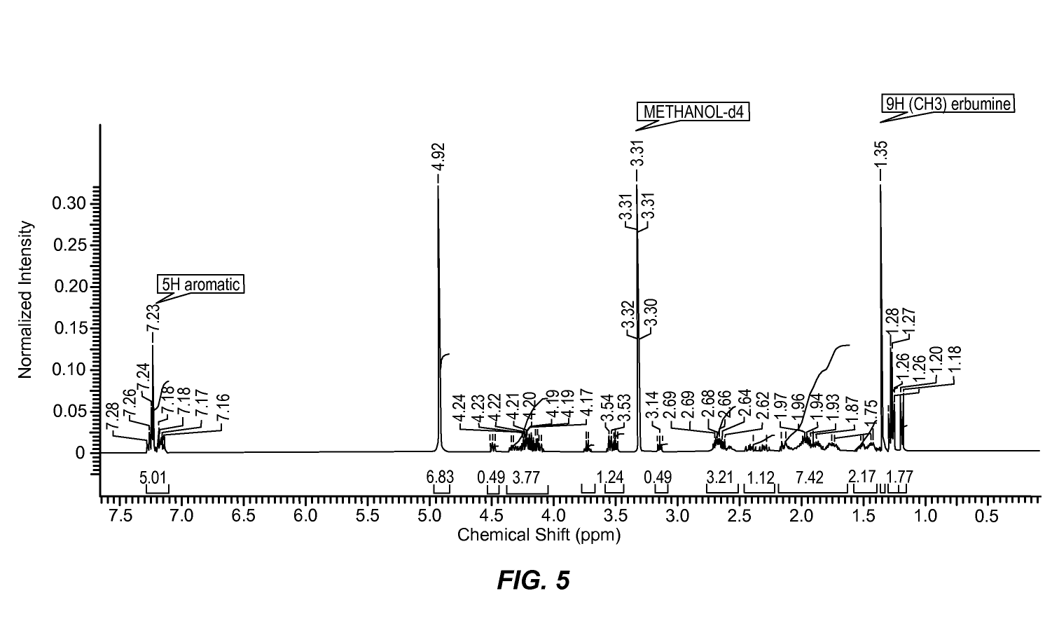

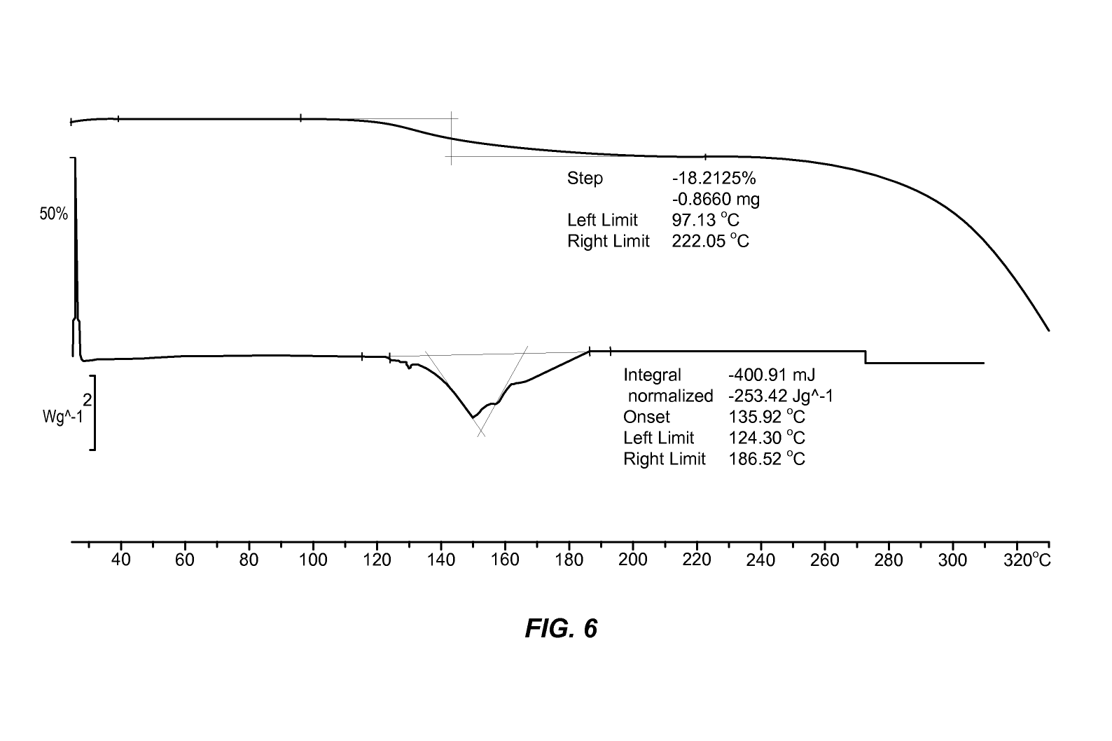

Ramipril (free acid) (12.13 g) was added to a 500 mL jacketed vessel, and ethyl acetate was added (390 mL). The resultant suspension was heated to 40° C. for 25 minutes using overhead stirring. After this time, the resultant suspension was heated to 50° C. (measured internal temperature 46° C.), and all remaining solid dissolved. After 5 minutes, tert-butylamine (1.0 equivalent, 3.1 mL in 5 mL ethyl acetate) was added. Within minutes, precipitation occurred and stirring became difficult. The stirring was increased to 190 rpm and the mixture was cooled gradually to 10° C. over 1 hour. After 15 minutes at 10° C., the solid precipitate was filtered under vacuum and washed out with ethyl acetate (30 mL). The precipitate cake was washed with 40 mL of ethyl acetate, air dried for 30 minutes, and oven dried at 25° C. under reduced pressure for 18 hours to provide 9.60 g (67%) of a Form 1 ramipril-erbumine salt whose X-ray (powder) diffraction pattern is depicted in FIG. 1 (see peak summary below), IR spectrum is depicted in FIG. 2 (see peak summary below), ¹H-NMR (methanol-d₄) is depicted in FIG. 5 and TGA (upper plot) and DSC (lower plot) are depicted in FIG. 6. The ramipril-erbumine salt made by this method was crystalline.

IR Summary

| Peak (cm⁻¹) | % T |
|---|---|
| 3315 | 98.2 |
| 2961 | 86.1 |
| 2915 | 86.5 |
| 2844 | 86.5 |
| 2749 | 89.1 |
| 2640 | 88.1 |
| 2559 | 89.6 |
| 2232 | 94.6 |
| 1722 | 67.5 |
| 1635 | 60.6 |
| 1569 | 62.6 |
| 1499 | 91.2 |
| 1474 | 91.1 |
| 1451 | 83.5 |
| 1419 | 73.9 |
| 1392 | 63.7 |
| 1365 | 81.1 |
| 1319 | 84.9 |
| 1271 | 74.8 |
| 1229 | 77.3 |
| 1195 | 86.0 |
| 1145 | 90.5 |
| 1111 | 87.1 |
| 1058 | 80.8 |
| 1036 | 78.3 |
| 968 | 94.7 |
| 920 | 93.1 |
| 858 | 91.5 |
| 835 | 93.3 |
| 793 | 91.6 |
| 745 | 74.6 |
| 720 | 84.2 |
| 705 | 70.0 |

Form 1 XRPD Summary

| Peak (2-Theta °) | Intensity (%) |
|---|---|
| 7.2 | 79.7 |
| 9.4 | 18.6 |
| 9.8 | 9.0 |
| 10.6 | 7.8 |
| 10.8 | 11.2 |
| 12.5 | 9.0 |
| 14.5 | 67.7 |
| 15.2 | 34.7 |
| 16.5 | 48.3 |
| 17.0 | 17.0 |
| 18.1 | 100.0 |
| 19.0 | 18.7 |
| 19.6 | 21.6 |
| 20.0 | 24.8 |
| 20.5 | 20.6 |
| 21.4 | 65.1 |
| 22.9 | 83.9 |
| 24.1 | 17.0 |
| 24.8 | 19.9 |
| 25.5 | 78.0 |
| 26.2 | 18.2 |

Ramipril-Erbumine Salt

Method B

Ramipril (1.913 g) was added to butanone (10 mL) and the resultant solution was brought to reflux. To the resultant clear solution was added dropwise tert-butylamine (0.5 mL). The solution was stirred at reflux for 10 minutes, resulting in the formation of a thick white precipitate. Butanone (20 mL) was added and the resultant clear solution was refluxed for a further 20 minutes. The solution was allowed to cool to room temperature. The resultant crystals were collected by filtration, washed with butanone and dried at reduced pressure to provide a ramipril-erbumine salt whose melting point was 140-146° C.

Ramipril-Erbumine Salt

Method C

Ramipril (1.98 g) was added to ethyl acetate (20 mL), and the resultant suspension was heated to reflux. A further quantity of ethyl acetate (15 mL) was added, and tert-butylamine (2.8 mL) was added dropwise to the refluxing solution. After a short period of time a white precipitate formed. Ethyl acetate (120 mL) was added to dissolve the precipitate, and the solution was kept at reflux for 20 minutes. The resultant clear solution was filtered hot and the clear filtrate was allowed to cool to room temperature and left overnight. The resultant white product was filtered, washed with ethyl acetate and dried under reduced pressure to provide a ramipril-erbumine salt whose melting point was 126-131° C.

Ramipril-Erbumine Salt

Method D

Ramipril (2.1 g) was added to 2,2-dimethoxypropane (65 mL) at reflux temperature. To the resultant clear solution, tert-butyl amine (0.2 g) in 2,2-dimethoxypropane (10 mL) was added slowly. The solution was refluxed for 10 minutes and cooled to room temperature. The resultant product was collected by filtration and dried under vacuum at 21° C. to provide a ramipril-erbumine salt as a white crystalline solid whose melting point was 140-146° C.

Ramipril-Erbumine Salt

Method E

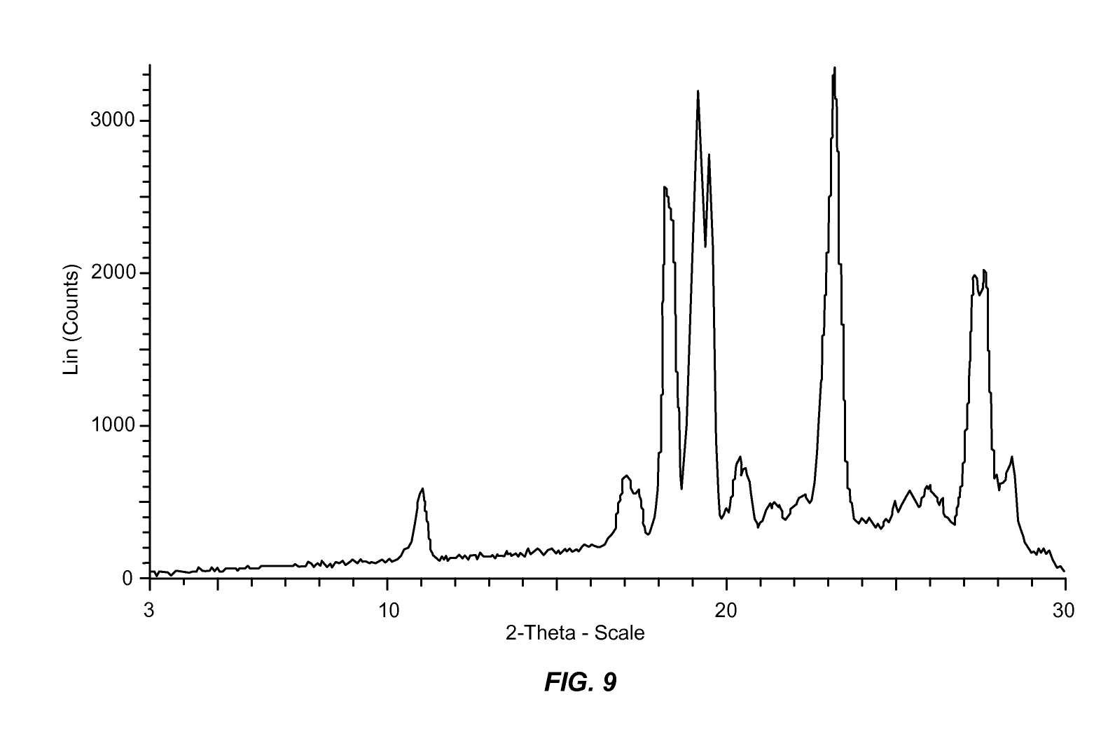

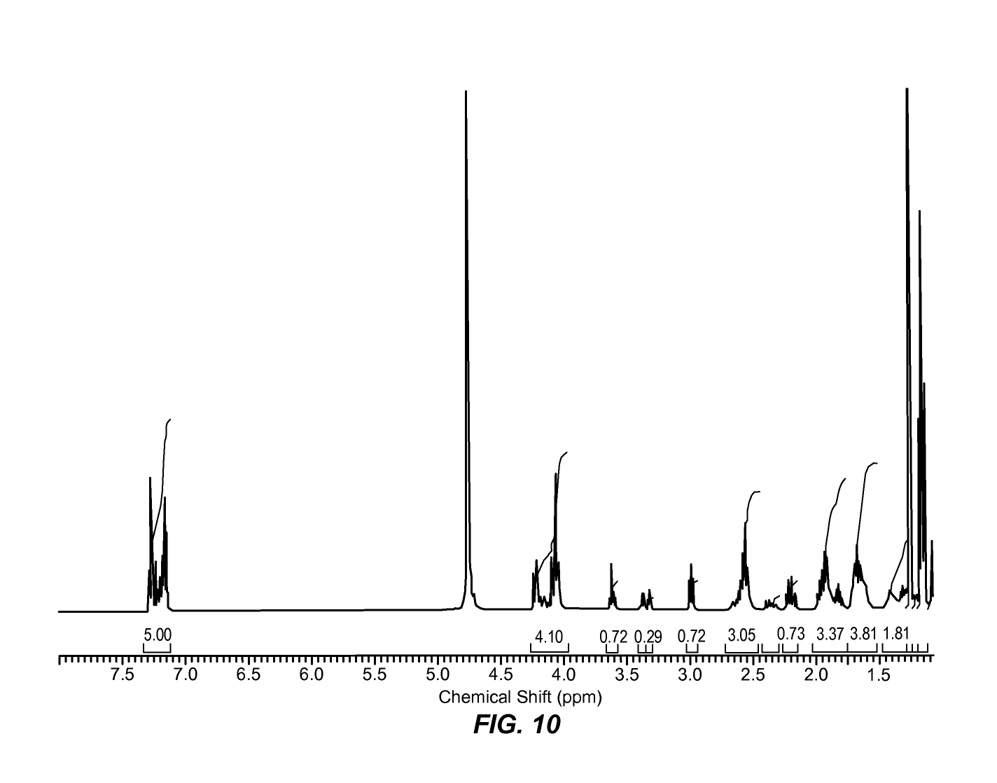

Ramipril free acid (1 g) was added to ethyl acetate (10 mL), stirred, and warmed to 50° C. tert-Butyl amine (1 equivalent) was added. After a few minutes, a white solid precipitated. Ethyl acetate (20 mL) was added to aid the stirring and the temperature was increased to 70° C. The precipitate did not completely dissolve. The suspension was cooled to room temperature, filtered, and dried at 25° C./5 mbar for 2 hours, yielding Form 2 ramipril-erbumine salt (0.897 g) whose X-ray (powder) diffraction pattern is depicted in FIG. 9 (see peak summary below) and whose ¹H-NMR spectrum in D₂O is depicted in FIG. 10.

Form 2 XRPD Summary

| Peak (2-Theta °) | Intensity (%) |
|---|---|
| 11.0 | 17.2 |
| 17.1 | 19.8 |
| 17.4 | 16.8 |
| 18.3 | 76.7 |
| 19.3 | 95.8 |
| 19.6 | 83.2 |
| 20.4 | 23.1 |
| 20.7 | 20.8 |
| 21.5 | 13.9 |
| 22.4 | 16 |
| 23.3 | 100 |
| 25.0 | 14.5 |
| 25.5 | 16.5 |
| 26.0 | 17.8 |
| 26.4 | 15.1 |
| 27.4 | 59.2 |
| 27.7 | 60.3 |
| 28.4 | 23.1 |

Ramipril-Ammonia Salt

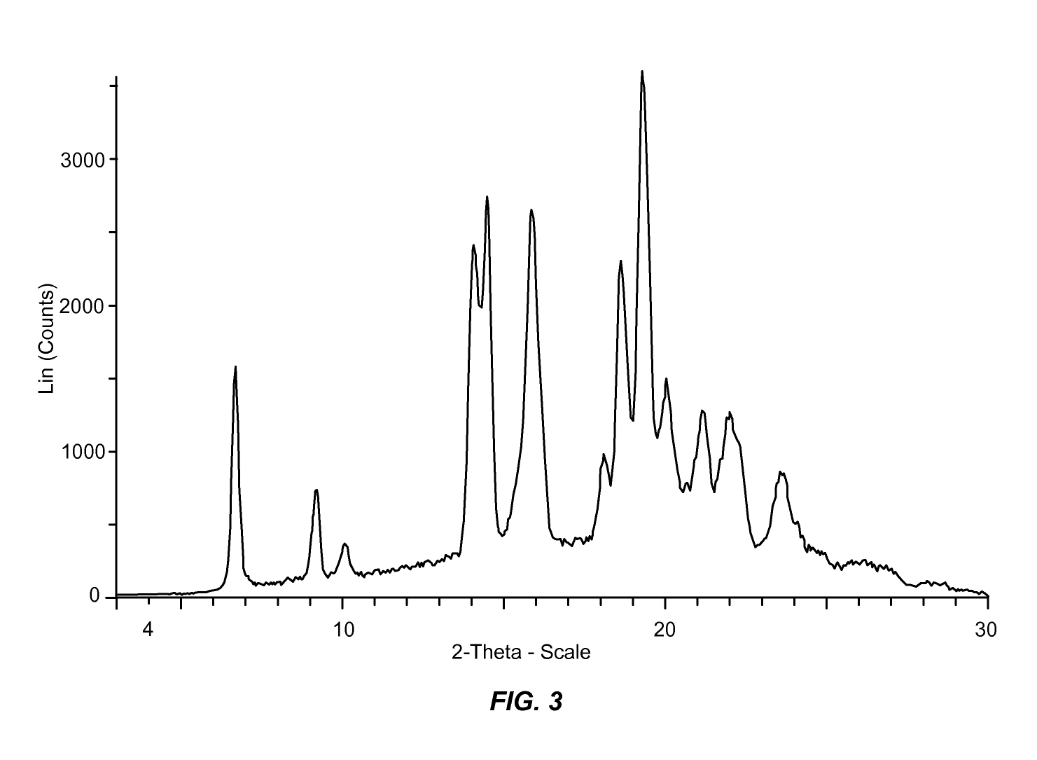

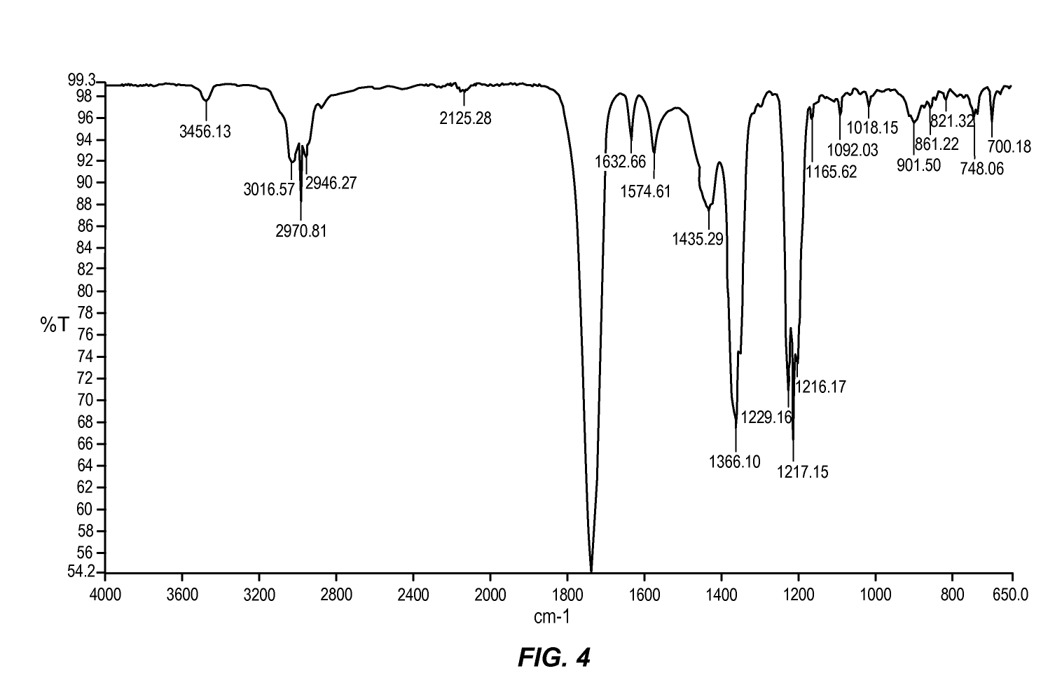

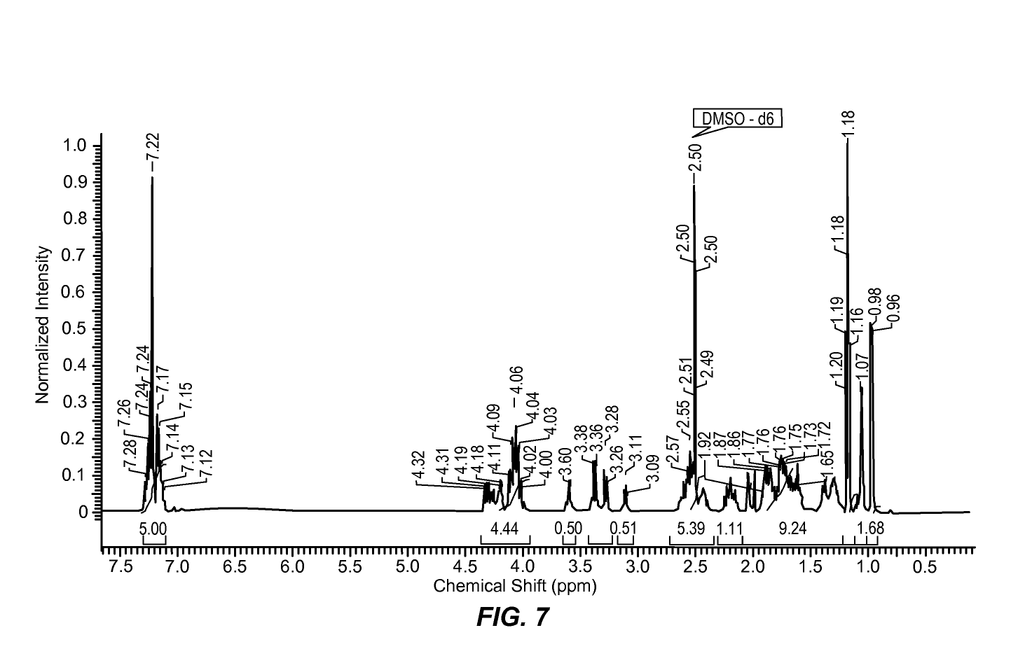

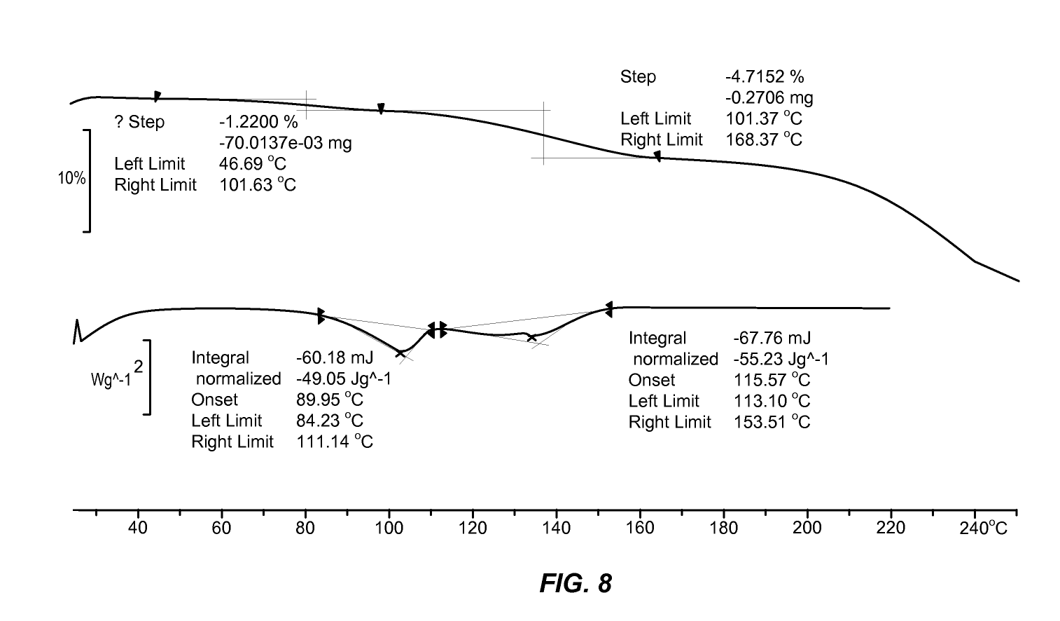

Ramipril (14.95 g) was added to a 1 L round bottomed flask, and ethyl acetate was added (250 mL). The resultant suspension was heated to 40° C. for 10 minutes. After this time a suspension remained. An additional 50 mL of Ethyl acetate was added, and the resultant solution was heated to 50° C. (measured internal temperature 48° C.). The solution was then allowed to cool to 37.5° C., and ammonia (36 mL, 2M in ethanol) was added in two portions (1×20 mL, 1×16 mL). A drop to 32.5° C. was then observed for the reaction mixture and precipitation occurred with 30 seconds. The resultant suspension was stirred for one hour, and the solid was filtered under vacuum and washed out with cold ethyl acetate (40 mL) and cold n-heptane (40 mL). The solid cake was air dried for 2 hours, then oven dried at 25° C. under reduced pressure for 60 hours to provide 8.80 g (57%) of ramipril-ammonia salt, whose X-ray (powder) diffraction pattern is depicted in FIG. 3 (see peak summary below), IR spectrum is depicted in FIG. 4 (see peak summary below), ¹H-NMR (methanol-d₄) is depicted in FIG. 7 and TGA (upper plot) and DSC (lower plot) are depicted in FIG. 8. The ramipril-ammonia salt made by this method was crystalline.

IR Summary

| Peak (cm⁻¹) | % T |
|---|---|
| 3456 | 97.6 |
| 3017 | 91.8 |
| 2971 | 88.2 |
| 2946 | 92.4 |
| 2125 | 98.4 |
| 1739 | 54.2 |
| 1633 | 93.9 |
| 1575 | 92.8 |
| 1435 | 87.5 |
| 1366 | 67.5 |
| 1229 | 70.7 |
| 1217 | 66.3 |
| 1206 | 73.3 |
| 1166 | 95.9 |
| 1092 | 96.2 |
| 1018 | 97.0 |
| 902 | 95.6 |
| 861 | 96.9 |
| 821 | 97.8 |
| 748 | 96.2 |
| 700 | 95.7 |

XRPD Summary

| Peak (2-Theta °) | Intensity (%) |
|---|---|
| 6.7 | 43.5 |
| 9.2 | 20.1 |
| 10.1 | 9.9 |
| 14.1 | 66.8 |
| 14.5 | 75.9 |
| 15.9 | 73.1 |
| 18.1 | 27.0 |
| 18.7 | 63.8 |
| 19.4 | 100.0 |
| 20.0 | 41.3 |
| 21.2 | 35.2 |
| 22.0 | 34.9 |
| 22.3 | 29.3 |
| 23.6 | 23.2 |

Ramipril-Triethylamine Salt

Ramipril (1.004 g) was added to butanone (10 mL), and the resultant solution was brought to reflux. To the resultant clear solution was added dropwise triethylamine (0.33 mL). The solution was stirred at reflux for 30 minutes and allowed to cool to room temperature overnight. The solvents were removed by distillation at reduced pressure. Ethyl acetate (10 mL) was added, and the distillation was repeated to afford a ramipril-triethylamine salt as an oil.

Biological Assays

Determination of the Effect of a Ramipril-Amine Salt on In Vivo Models of Reperfusion Injury

The efficacy of a ramipril-amine salt in a mouse model of ischemic and reperfused gut can be determined according to the method described in Liaudet et al., Shock 2000, 14(2): 134-41.

In another set of experiments, the effect of a ramipril-amine salt in a rat model of middle cerebral artery occlusion/reperfusion can be assayed as described in Abdelkarim et al, Int. J. MoI Med. 2001, 7(3):255-60.

Determination of the Effect of a Ramipril-Amine Salt on In Vivo Models of Hypertension

The efficacy of a ramipril-amine salt in a mouse model of hypertension can be determined according to the methods described in Badyal et al. Indian J. of Pharmacology, 2003, 35:349-362.

Determination of the Effect of a Ramipril-Amine Salt on In Vivo Models of Cardiovascular Disease

The efficacy of a ramipril-amine salt in a mouse model of cardiovascular disease can be determined according to the methods described in Rusell et al. Cardiovasc Pathol. 2006, 15(6):318-30.

Determination of the Effect of a Ramipril-Amine Salt in an In Vivo Model of Diabetes Mellitus

The anti-diabetic effect of a ramipril-amine salt can be determined using a single high-dose streptozotocin model of diabetes mellitus, which can be used as conducted as described in Mabley et al., Br. J. Pharmacol. 2001, 133(6):909-9; and Soriano et al., Nat. Med. 2001, 7(1):108-13.

We claim:

1. A ramipril-amine salt.

2. The salt of claim 1, wherein the amine is ethanolamine, triethanolamine, erbumine, ammonia, triethylamine, meglumine, ethylamine diamine, choline, procaine or benzathine.

3. The salt of claim 1, having the formula X(NR¹R²R³), wherein X is ramipril and each of R¹, R² and R³ is independently selected from H, unsubstituted or substituted alkyl, unsubstituted or substituted cycloalkyl, unsubstituted or substituted aryl, unsubstituted or substituted heteroaryl, or unsubstituted or substituted heterocycloalkyl

4. The salt of claim 1, having the formula X(NR¹R²R³R⁴) wherein X is ramipril and each of R¹, R², R³ and R⁴ is independently selected from H, unsubstituted or substituted alkyl, unsubstituted or substituted cycloalkyl, unsubstituted or substituted aryl, unsubstituted or substituted heteroaryl, or unsubstituted or substituted heterocycloalkyl.

5. The salt of claim 1, wherein the amine is not dicyclohexylamine or meglumine.

6. The salt of claim 1, wherein the salt is substantially free or ramipril (free acid) or amine (free base).

7. The salt of claim 1, in crystalline form.

8. The salt of claim 1, in non-crystalline form.

9. A composition comprising a therapeutically effective amount of ramipril-amine salt and a pharmaceutically acceptable carrier.

10. The composition of claim 9, further comprising another therapeutic agent.

11. The composition of claim 10, wherein the other therapeutic agent is a diuretic, a statin, a calcium channel blocker, a statin, a calcium channel blocker, an antiinflammatory agent, an anti-renal failure agent, an anti-diabetic agent, an anti-cardiovascular disease agent, an opioid analgesic agent, a non-opioid analgesic agent, an antibiotic, an antiemetic, an anti fungal agent, an antidepressant, an immunomodulatory agent, a cytokine, a hormone, or a β-interferon.

12. A method for treating a cardiovascular disorder comprising administering to a subject in need thereof a therapeutically effective amount of a ramipril-amine salt.

13. The method of claim 12, further comprising administering another therapeutic agent.

14. A method for treating an ischemic condition, comprising administering to a subject in need thereof a therapeutically effective amount of a ramipril-amine salt.

15. The method of claim 14, further comprising administering another therapeutic agent.

16. A method for treating renal failure, comprising administering to a subject in need thereof a therapeutically effective amount of a ramipril-amine salt.

17. The method of claim 16, further comprising administering another therapeutic agent.

18. A method for treating diabetes mellitus or a diabetic condition, comprising administering to a subject in need thereof a therapeutically effective amount of a ramipril-amine salt.

19. The method of claim 18, further comprising administering another therapeutic agent.

20. A method for reducing the incidence of recurrence or severity of a symptom of a cardiovascular disorder, an ischemic condition, renal failure, diabetes mellitus or a diabetic condition, comprising administering to a subject in need thereof a therapeutically effective amount of a ramipril-amine salt.

21. The method of claim 20, further comprising administering another therapeutic agent.
